(19) United States
(12) Patent Application Publication
(10) Pub. No.: US 2003/0004574 A1
(43) Pub. Date: Jan. 2, 2003

(54) Disc and annulus augmentation using biologic tissue

(76) Inventor: Bret A. Ferree, Cincinnati, OH (US)

Correspondence Address: John G. Posa, Gifford, Krass, Groh, Sprinkle,, Anderson & Citkowski, PC, 280 N. Old Woodward Ave., Suite 400, Birmingham, MI, 48009, US

(21) Appl. No.: 10/225,739

(22) Filed: Aug. 22, 2002

Related U.S. Application Data
(63) Continuation-in-part of application No. 10/120,763, filed on Apr. 11, 2002.
Application No. 10/120,763 is a continuation-in-part of application No. 09/807,820, filed on Apr. 19, 2001.
Application No. 09/807,820 is a 371 of international application No. PCT/US00/14708, filed on May 30, 2000.
Application No. 09/807,820 is a 371 of international application No. 09/638,241, filed on Aug. 14, 2000.
Application No. 09/807,820 is a 371 of international application No. 09/454,908, filed on Dec. 3, 1999.
Application No. 09/807,820 is a 371 of international application No. 09/639,309, filed on Aug. 14, 2000, now Pat. No. 6,419,702.
Application No. 09/807,820 is a 371 of international application No. 09/690,536, filed on Oct. 16, 2000, now Pat. No. 6,371,990.
Application No. 09/690,536 is a continuation-in-part of application No. 09/638,726, filed on Aug. 14, 2000, now Pat. No. 6,340,369.
Application No. 09/690,536 is a continuation-in-part of application No. 09/415,382, filed on Oct. 8, 1999, now Pat. No. 6,419,704.

(51) Int. Cl. A61F 2/44

(52) U.S. Cl. 623/17.12

(57) ABSTRACT

Devices to augment and/or fortify a human disc use a bag, body or other suitable enclosure fashioned from fascia, skin, or other applicable tissue from a living or recently deceased human or animal donor. The devices may aid the ingrowth of the patient's tissue and, in time, the patient's body may replace the transplanted tissue. The device exhibits a final volume sized to consume at least a portion of the intervertebral disc space, with the biologic tissue and filler material enabling the body to cyclically compress and expand in a manner similar to the disc material being replaced or augmented. Various filler materials may be used to impart an appropriate level of compressibility, including polymeric urethanes or other suitable elastomers, hydrogels, or biologic tissues. Devices according to the invention may be attached to the inside and/or outside of the annulus by stitches, staples, adhesives, or other suitable techniques. Alternatively, the device may be attached to the vertebra above and below the disc by screws, staples, tacks, or porous material for bone ingrowth such as titanium. Other methods of attachment to the annulus or vertebrae would also be acceptable if the overall goals of the invention are otherwise achieved.

### REFERENCE TO RELATED APPLICATIONS

This application is a continuation-in-part of U.S. patent application Ser. No. 10/120,763, filed Apr. 11, 2002, which is a continuation-in-part of U.S. patent application Ser. No. 09/807,820, which is a 371 of PCT/US00/14708, filed May 30, 2000; Ser. Nos. 09/638,241, filed Aug. 14, 2000; and 09/454,908, filed Dec. 3, 1999; and 09/639,309, filed Aug. 14, 2000, now U.S. Pat. No. 6,419,702; and 09/690,536, filed Oct. 16, 2000, now U.S. Pat. No. 6,371,990, which is a continuation-in-part of U.S. patent application Ser. Nos. 09/638,726, filed Aug. 14, 2000, now U.S. Pat. No. 6,340,369; and 09/415,382, filed Oct. 8, 1999, now U.S. Pat. No. 6,419,704, the entire content of each application being incorporated herein by reference.

### FIELD OF THE INVENTION

This invention relates generally to human spinal surgery and, in particular, to methods and apparatus associated with annulus fibrosis augmentation, and partial and full disc replacement.

### BACKGROUND OF THE INVENTION

According to human anatomy, spinal function is dependent upon the intervertebral disc and the facet joints. In a sense, the annulus fibrosis, nucleus pulpous, and the facet joints form the legs of a three-legged stool.

The annulus is formed of 10 to 60 fibrous bands which serve to control vertebral motion. One half of the bands tighten to check motion when the vertebra above or below the disc are turned in either direction. Restoring disc height returns tension to the annular noted in the prosthetic disc patent application. In addition, restoring annular tension decreases annular protrusion into the spinal canal or neural foramen. Thus, decreasing annular protrusion may eliminate pressure on the spinal cord or nerve roots.

At times the rotational, translational, and axial compression forces exceed the strength of the annular fibers. The excessive forces tear the annular fibers. A single event can tear one band to all the bands. Subsequent tears can connect to previous tears of a few bands resulting in a hole through the entire annulus fibrosis. Holes through the entire annulus fibrosis can result in extrusion of the nucleus pulpous. Extrusion of the nucleus pulpous is referred to as a “herniated disc.” Disc herniation can result in back pan, neck pain, arm pain, leg pain, nerve or spinal cord injury, or a combination of the above.

Since the annulus is innervated with pain fibers, acute annular tears without herniation of the nucleus can be painful. Unfortunately, the annular tears often do not heal completely. The chronic tears can result in neck pain, back pain, shoulder pain, buttock pain, or thigh pain. The chronic tears weaken the annulus fibrosis predisposing the disc to herniation or additional annular tears. My U.S. Pat. No. 6,340,369, entitled “Methods and Apparatus for Treating Disc Herniation,” and U.S. Pat. No. 6,419,704, entitled “Artificial Intervertebral Disc Replacement” describe methods and apparatus for occluding annular defects. These patents, incorporated herein by reference, also discuss spinal anatomy, spinal physiology, disc degeneration, surgical and non-surgical treatments of disc disease, and the advantages of prosthetic disc replacement.

To restore disc height resulting, for example, from degenerative disease, prosthetic discs are used to replace only the nucleus pulpous. However, prosthetic replacement of the nucleus pulpous alone risks future problems arising from annular tears. Patients may continue to complain of pain from the stresses placed onto the weakened annulus. Secondly, tears of the annulus could result in extrusion of the prosthetic nucleus. In addition, remaining nucleus pulpous could herniate through annular tears.

Some prosthetic disc designs attempt to replace nucleus and annular functions. In general, these designs attach the prosthetic disc to the vertebrae. Many of the techniques in this area attach the prosthetic disc to the end plates of the vertebrae with screws, spikes, flanges, or porous surfaces for bone ingrowth. My U.S. Pat. Nos. 6,245,107 and 6,419,704 describe methods and devices to assist the annulus in retaining remaining nucleus pulpous and a prosthetic nucleus. The entire contents of these applications are also incorporated herein by reference.

The need remains, however, for a more biologically compatible disc/annulus augmentation technique. Ideally, such an improved technique would aid with ingrowth while, at the same time, replace augmented tissue.

### SUMMARY OF THE INVENTION

This invention broadly resides in devices to augment and/or fortify a human disc using human or animal tissue. In the preferred embodiments, a bag, sealed body, or the like, is fashioned from fascia, skin, or other applicable tissue from a living or recently deceased human or animal donor, and used to supplement the annulus fibrosis or partially or entirely replace a disc. Such devices may aid the ingrowth of the patient's tissue and, in time, the patient's body may replace the transplanted tissue.

Broadly, the invention is used to construct a shaped body having a final volume sized to consume at least a portion of the intervertebral disc space, with the biologic tissue and filler material enabling the body to cyclically compress and expand in a manner similar to the disc material being replaced or augmented. Various filler materials may be used to impart an appropriate level of compressibility, including polymeric urethanes, elastomers, or other biologic tissues.

In any case, the body may assume some form of collapsed state permitting easier insertion, and a final state having superior and inferior surfaces preferably conformal to the concavities of the vertebral endplates. The superior and inferior surfaces may accordingly be convex, and may further include grooves, spikes, or other protrusions to maintain the body within the intervertebral space. The body may further be wedge-shaped to help restore or maintain lordosis, particularly if the prosthesis is introduced into the cervical or lumbar regions of the spine.

Devices according to the invention may be attached to the inside and/or outside of the annulus by stitches, staples, adhesives, or other suitable techniques. Alternatively, the device may be attached to the vertebra above and below the disc by screws, staples, tacks, or porous material for bone ingrowth such as titanium. Other methods of attachment to the annulus or vertebrae would also be acceptable if the overall goals of the invention are otherwise achieved.

### BRIEF DESCRIPTION OF THE DRAWINGS

FIG. 1A is a simplified drawing illustrating the implantation of one prosthesis according to the invention as viewed upwardly toward the head of the recipient;

FIG. 1B is a simplified drawing of the single prosthesis of FIG. 1A as seen from the back;

FIG. 1C is a simplified drawing of the single prosthesis embodiment of FIG. 1A, as viewed from the side;

FIG. 2A is a simplified drawing of a disc replacement according to the invention utilizing two prosthesis per disc as viewed upwardly toward the head of the recipient;

FIG. 2B is a simplified drawing of the two prosthesis embodiment of FIG. 2A as viewed front to back;

FIG. 2C is a simplified drawing of the embodiment of FIG. 2A as viewed from the side;

FIG. 3A is a simplified drawing of an alternative configuration utilizing two prosthesis per disc placed laterally;

FIG. 3B is a simplified drawing of the lateral placement of FIG. 3A as viewed from the back;

FIG. 3C is a simplified drawing of the lateral placement of FIG. 3A as viewed front to side;

FIG. 4A is an oblique representation of the way in which one or more flaps may be used to insert a prosthesis into a retainer according to the invention;

FIG. 4B is a drawing of the arrangement of FIG. 4A, but with the annular flaps opened;

FIG. 4C is a drawing which illustrates the alternative use of a band to close off one or more annular flaps used to introduce an intravertebral disc replacement according to the invention;

FIG. 4D which is a drawing which furthers the configuration shown in FIG. 4C, wherein a second intervertebral disc replacement is being introduced;

FIG. 4E is a drawing which subsequent to that of FIG. 4D, wherein the band is used to close off a pair of annular flaps;

FIG. 4F is a drawing which shows how a flexible patch or retaining pieces may be used to close off an annular flap according to the invention;

FIG. 4G which shows the flexible material and retaining pieces from a side-view perspective;

FIG. 4H illustrates an alternative use of crisscross bands for use in annular flap closure;

FIG. 5 is a drawing which shows the way in which a sealed body may be constructed from human or animal tissue;

FIG. 6 is a perspective drawing of an alternative embodiment of a tissue bag or sealed body;

FIG. 7 is a drawing which shows the way pieces of tissue may be folded over and sewn with a gap to receive filler material;

FIG. 8A is a top-view drawing which shows how multiple disc replacement components may be interlocked according to the invention;

FIG. 8B illustrates a different interlocking scheme;

FIG. 8C illustrates yet a different interlocking scheme having one or more plugs and receptacles; and

FIG. 8D illustrates yet a further technique for interconnecting multiple intervertebral disc replacement parts according to the invention.

### DETAILED DESCRIPTION OF THE INVENTION

This invention resides in prosthetic disc replacement and annulus augmentation devices using human or animal tissue which exhibit a desirable level of compression along the spine. In the preferred embodiments, a bag or sealed body is fashioned from fascia, skin, or other applicable tissue from a living or recently deceased human or animal donor, and used to supplement the annulus fibrosis or partially or entirely replace a disc. Such devices may aid the ingrowth of the patient's tissue and, in time, the patient's body may replace the transplanted tissue.

Broadly, and in general terms, a device according to the invention comprises a bag or shaped body which either contains a material, or is filled with a material, enabling the body to cyclically compress and expand in a manner similar to natural disc material. Such materials may be natural, synthetic, or a combination thereof. For example, the enclosure may be filled with biologic tissue from the patient or other human/animal donor. Such tissue may include, though not limited to, disc tissue, tendon, ligament, or meniscus. In these and in other situations disclosed herein, the bag may be porous to allow for fluids to pass through the enclosure.

In an inflatable embodiment, a prosthetic disc according to the invention is sealed to be filled with air, oxygen or another suitable gas or gas mixtures. The body may also be filled with a liquid, oil, saline solution, elastomer, or gel. Hydrogels used in this embodiment are preferably sealed in the prosthetic disc in a dehydrated state. Once the prosthetic disc is placed in the spine, a liquid is added through the valve or directly through the prosthetic disc (e.g., by a needle and a syringe) to hydrate the hydrogel. U.S. Pat. Nos. 5,047,055 and 5,192,326 provide a listing of hydrogels, at least certain of which are applicable to this invention. One advantage of hydrating the gel in a sealed body is that even if pinholes form in the body, the device will still function properly, assuming biocompatible filler materials are used.

As an alternative, the body may contain one or more liquids or solids which, when mixed, produce a gas, thereby filling the body. For example, baking soda and vinegar may be used or other materials which offer a greater ratio of starting materials to the final volume, including expandable foams. In the event that a liquid is one of the components, it may be contained in an ampoule of some kind which is opened upon insertion of the body into the disc space, thereby allowing the constituent materials to mix. In a different arrangement, the body may include some form of structure with a window or other port which becomes compromised in the presence of externally supplied energy in the form of ultrasound, heat, etc., thereby allowing a foam to expand, reactants to mix, and so forth.

According to a different preferred embodiment, the prosthetic disc does not contain a cavity, but is constructed of a biologically compatible yet compressible material such as silicone or rubber with a cover formed of human or animal tissue. In this embodiment, the prosthesis would not be inflated or imbibe fluid to expand. Rather, the prosthesis would preferably be compressed or deformed prior to insertion into the disc space.

Regardless of the embodiment, the prosthetic disc according to the invention would be inserted through the annulus fibrosis in a surgical procedure. The surgeon would cut a flap or hole in the annulus, and the degenerated nucleus pulpous would be removed according to the standard techniques. One, two, or more prosthesis according to the invention would then be introduced into the disc space, depending upon the location in the body, patient physiology, and so forth.

Although the size examples shown in the drawings may be implied as applicable to human lumbar disc, the prosthesis according to the invention may also be provided for cervical and thoracic discs as well as other joints of the body or animals, through appropriate geometrical scaling. In addition, although it is implied that when multiple prosthesis are used, the same embodiment of the prosthesis would be introduced into the disc space, this is not necessarily always the case, since the various embodiments disclosed herein may be mixed and matched, even within the same disc space, depending upon the physical arrangement.

The prostheses could be inserted through the posterior, anterior, or lateral portion of the intervertebral disc by standard surgical procedures. In each case, the prosthesis would come in various sizes to accommodate different size discs. If two prostheses are used in one disc space, each prosthesis would measure approximately 10-30 mm×10-20 mm×5-20 mm. If one prosthesis is used, it would measure approximately 10-30 mm×20-40 mm×5-20 mm. The prostheses could be used in cervical, thoracic, or lumbar discs of animals and humans. In addition the device could be used in other joints, including the ankle.

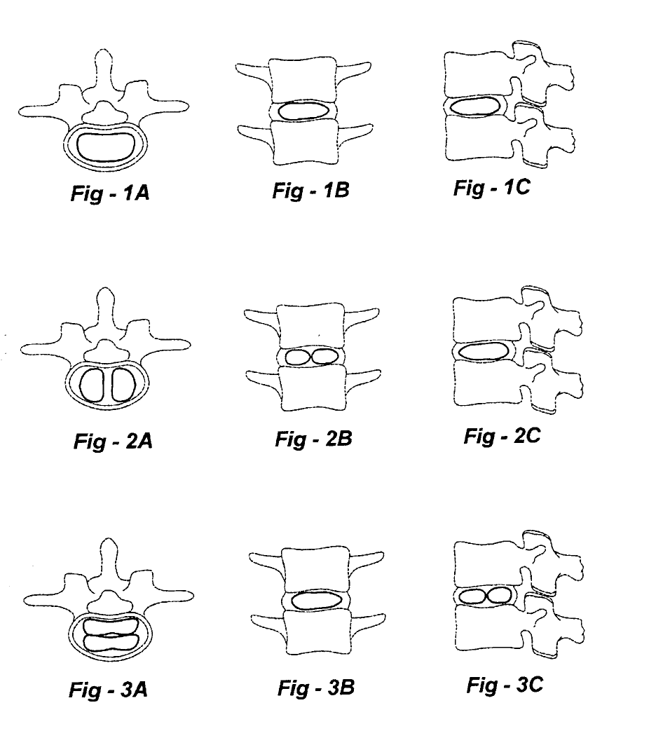

FIG. 1A is a simplified drawing which illustrates the implantation of one prosthesis according to the invention as viewed upwardly toward the head of the recipient. FIG. 1B is a simplified drawing of the single prosthesis as seen from the back, and FIG. 1C is a view from the side. FIG. 2A is a simplified drawing of the invention utilizing two prosthesis per disc as viewed upwardly toward the head of the recipient. FIG. 2B is a simplified drawing as viewed front to back and FIG. 2C is a simplified drawing as viewed from the side. The prosthesis may be placed front to back or back to front, depending upon if entry is made through the back or abdomen of the patient. FIG. 3A is a simplified drawing of an alternative configuration utilizing two prosthesis per disc placed laterally. FIG. 3B is a view from the back, and FIG. 3C is a simplified drawing of a lateral placement as viewed front to side.

Particularly when a single prosthesis is used, it will preferably feature convex superior and inferior surfaces so as to conform to the concavities of the vertebral end plates. In addition, the prosthesis may be wedge-shaped, such that the anterior surface is taller than the posterior surface. Such a shape is particularly beneficial in restoring or maintaining lordosis in the cervical and lumbar region of the spine. In the event that a plurality of prostheses are positioned anterior to posterior, as shown in FIGS. 3A-3C, it may be advantageous to place thicker or less resilient devices anteriorally, with devices having a smaller cross-section or more compressible durometer posteriorally.

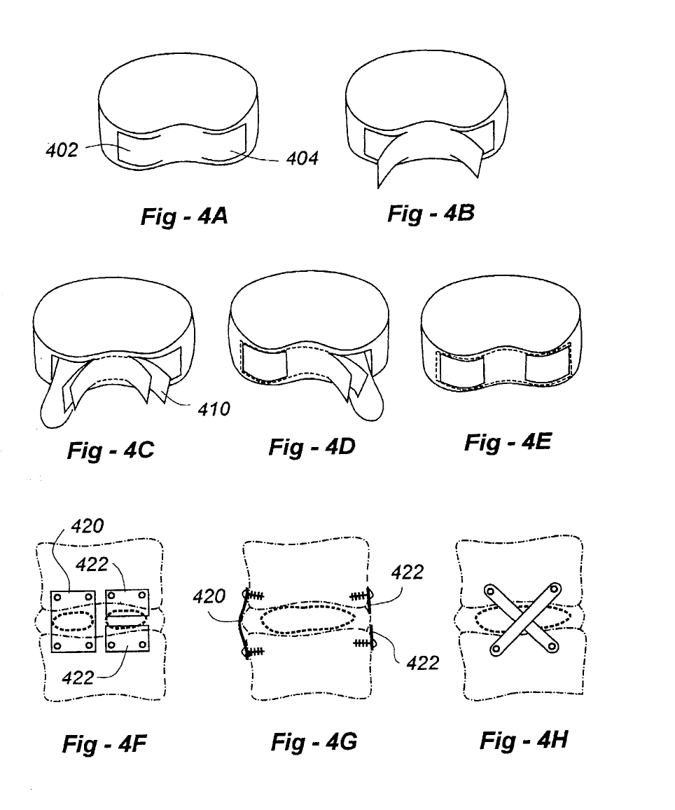

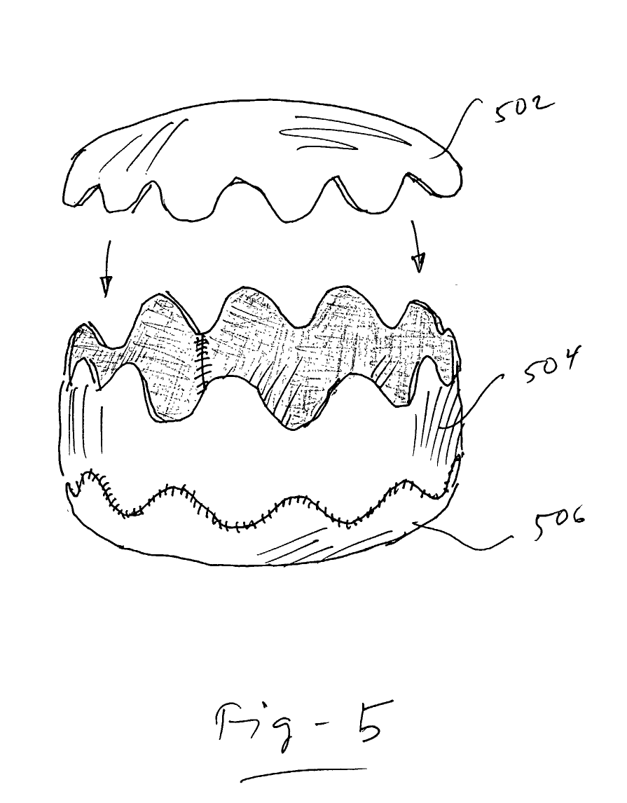

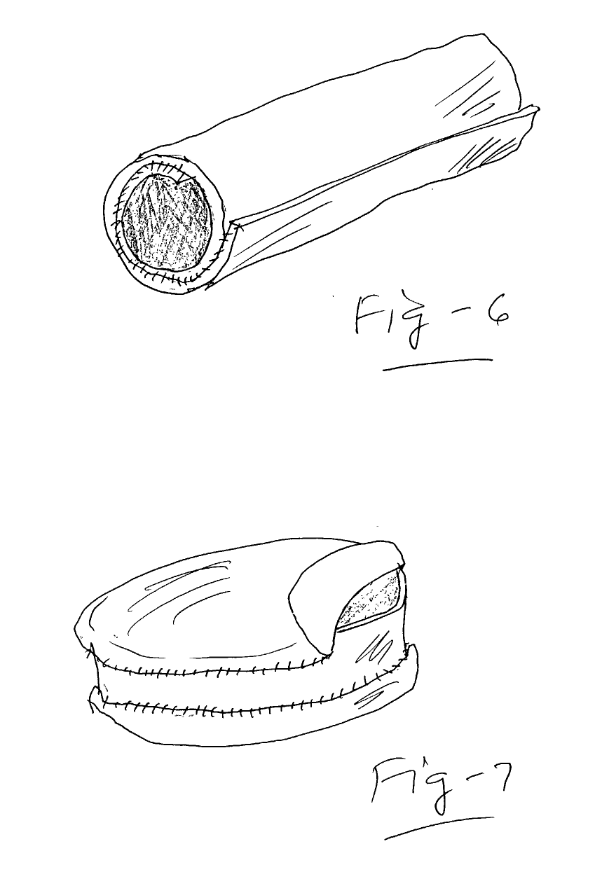

In the case of two prosthesis, two annular flaps 402 and 404 would preferably be created as shown in FIG. 4A. FIG. 4B is a drawing which shows the annular flaps in an open state. A prosthesis or prostheses may also be inserted through one annular flap. In addition, the prosthesis or prostheses may be inserted through the annular window that follows a procedure to remove a herniated nucleus pulpous. If annular flaps are formed, they may be sewn or sealed closed after insertion of the artificial disc or discs. The prosthetic disc or discs could restore a collapsed disc space by inflation of the prosthesis or prostheses. The vertebrae may also be distracted to restore normal disc height and aid the insertion of the prosthesis or prostheses, mechanically. As shown in FIGS. 4C-4E, a malleable band 410 of flexible plastic, metal or other material may be inserted through the annular flaps as shown, a material with a shape memory may be beneficial for such purpose. FIG. 4C shows a situation wherein a collapsed replacement is inserted into one of the two openings, and FIG. 4D shows a disc replacement member according to the invention being inserted into the other opening. FIG. 4E shows how the band of material 410 would be used to close both openings through suturing or other appropriate surgical techniques.

FIGS. 4F through 4H illustrate alternative approaches, wherein panels may be attached to adjacent vertebrae for the purpose of retaining disc replacement material. As shown in the front-view drawing of FIG. 4F, a flexible piece of material 420 may be attached to adjacent vertebrae in the form of a rectangular shape or cords. Such a material would permit normal movement of the spine, and may be attached to upper and lower vertebrae through any appropriate known technique for fixation. A cloth fabric, such as Gore-Tex® or Dacron®, a mesh screen such as nylon, or tissue from a live or recently deceased human or animal may be attached to the adjacent vertebrae as shown, allowing normal movement. Such a technique would be used primarily when the prosthetic disc is placed from an anterior approach to the spine, whether cervical thoracic or lumbar, and would help to restore normal annular function.

As an alternative to a flexible fabric, screen, or tissue one or more retaining members 422 may alternatively be utilized. Such a member, which may be plastic, metal or other suitable material, would be attached to one or both of the adjacent vertebrae as shown. FIG. 4G is a drawing which shows the fabric 420 in panels 422, as viewed from the side. FIG. 4H illustrates how materials may be applied in cris-cross fashion, in the form of bands, for example.

Mechanical distraction of the vertebra may also be used for disc replacement. U.S. Pat. No. 5,824,093, for example, describes an air jack that could be inserted through one of the flaps. Once the distraction is achieved, a prosthesis is inserted through the other annular flap. Air jacks of the type disclosed in the '093 patent may also be inserted through both annular flaps to achieve symmetric distraction. When properly distracted, one air jack may be deflated and removed. The first prosthesis would be inserted into the space formerly occupied by the air jack. After the first prosthesis is inserted, the second air jack would be deflated and removed. A second prosthesis would be inserted into the remaining disc space. A crank scissors jack could also be used to distract the vertebrae.

The intra-discal position of the prosthesis or prostheses may be maintained in a number of ways. First, the prosthesis diameter is larger in the center portion than the periphery. Second, the prosthesis expands after insertion through the annular opening. Third, the majority of annulus fibrosis is preserved. Fourth, the prosthesis exerts constant pressure on the adjacent vertebrae, securing a tight fit. Fifth, the vertebrae may be distracted so as to enlarge the disc space prior to inserting the prosthesis. When the distraction is released after prosthesis insertion, the tension placed on the annular fibers will serve to hold the prosthesis in position.

FIG. 5 is a drawing of an embodiment of the invention showing the way in which pieces of tissue 502, 504, 506 may be attached with sutures to form a bag/sealed body according to the invention. It will be appreciated that this is not the only way in which such tissue pieces may be joined, and that other constructions are possible. For example, the tissue may be cut into strips and woven together in basket form or “Chinese finger traps” sufficient to receive a filler. Alternatively, the tissue may be fashioned into a pouch with a first string or other type of closure.

It will also be appreciated that different types of tissue may be used, as appropriate including fascia from recently deceased human or animal donors, or skin from a recently deceived human or animal donor. The skin or other tissue pieces may be processed to make them more durable. For example, a tanning process may be used to create a leather-like material used in the hide processing industry. Generally, the use of chemicals and drying processes are well known to those in the area of tissue banking. While animal skins may alternatively be used, clearly the treatments involved would not use substances that could otherwise be harmful to the human recipient.

FIG. 6 is an alternative embodiment of the way in which an elongated tissue bag or sealed body may be constructed. In particular, pieces may be rolled or folded onto one another and sutured together to form a tube, particularly if multiple devices are used in the same disc space. As shown in FIG. 7, a gap may be left in a portion of the device through which to insert filler material, biologic or otherwise.

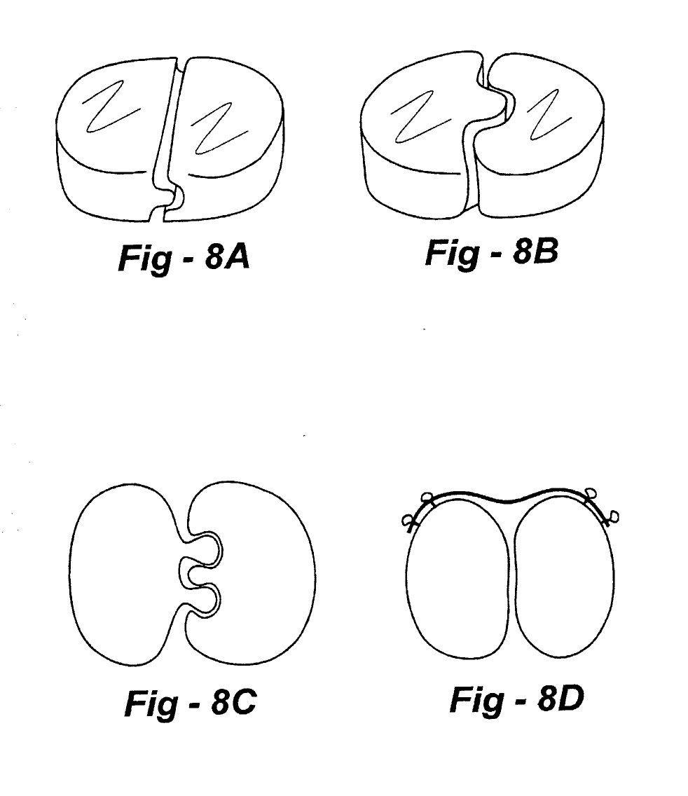

Generally, only one prosthetic disc would be placed into the disc space in the cervical region of the spine. If multiple prostheses are used, as shown in FIGS. 2A through 3C and 4F, the shapes may further include an interlocking structure to help hold them in place, at least relative to one another. FIG. 8A shows a lateral scheme for interlocking adjoining shaped bodies, whereas FIG. 8B illustrates a vertical arrangement much like puzzle pieces. Truly interlocking mechanisms may also be utilized, as shown in FIG. 8C, which incorporates knobs received by receptacles, and FIG. 8E, which illustrates a band of material which is fastened to adjacent devices through any appropriate form of fastener.

I claim:

1. An intervertebral disc replacement device, comprising:
an enclosure using tissue from a live or recently deceased human or animal donor, the enclosure being dimensioned to consume at least a portion of an intervertebral disc space; and
a filler material within the enclosure enabling the enabling the replacement to cyclically compress and expand in a manner similar to the disc material being replaced or augmented

2. The artificial disc replacement of claim 1, wherein tissue is fascia.

3. The artificial disc replacement of claim 1, wherein tissue is skin or skin-based.

4. The artificial disc replacement of claim 1, wherein the filler material is a gas, liquid, gel, biologic tissue, or a combination thereof.

5. The artificial disc replacement of claim 4, wherein the gel is a hydrogel.

6. The artificial disc replacement of claim 4, wherein the biologic tissue includes fascia, tendon, ligament, meniscus, disc tissue, or other biologic material from a human or animal donor.

7. The artificial disc replacement of claim 4, wherein the biologic tissue may contain living cells.

8. The artificial disc replacement of claim 1, wherein the sealed body includes at least one reactant which, when mixed with another reactant, results in an expansion to fill the body.

9. The artificial disc replacement of claim 1, further including a material disposed within the sealed shaped body which expands in the presence of externally applied energy.

10. The artificial disc replacement of claim 1, wherein the sealed body includes a surface with one or more features to maintain the body within the disc space.

11. The artificial disc replacement of claim 1, wherein the body assumes a convex or lordotic shape.

12. The artificial disc replacement of claim 1, including two or more of the enclosures appropriately sized to occupy the same disc space.

13. The artificial disc replacement of claim 1, wherein the enclosure further includes a moisture-permeable inner liner.
